(19) United States
(12) Patent Application Publication
(10) Pub. No.: US 2008/0234682 A1
(43) Pub. Date: Sep. 25, 2008

(54) Augmentation device for osteoporotic bone

(76) Inventor: Maxwell C. Park, Calabasas, CA (US)

Correspondence Address: Elliott N. Kramsky, Esq., Suite 400, 5850 Canoga Avenue, Woodland Hills, CA, 91367, US

(21) Appl. No.: 11/724,690

(22) Filed: Mar. 16, 2007

(51) Int. Cl. A61B 17/56; A61B 17/68; A61B 17/04

(52) U.S. Cl. 606/75; 606/300

(57) ABSTRACT

A bone augmentation device for anchoring an orthopaedic implant to bone tissue. An annular proximal section is rotatably joined to a distal section formed of a plurality of arcuate lower segments by flexures (integral embodiments) or a hinge (discrete embodiments). The interior of the annular proximal section is coaxial with the plurality of lower segments in the closed configuration of the distal section. When a surgical implant penetrates the device the lower segments, whose free ends are barbed, are rotated outwardly to either contact the endosteal side of the cortical bone or to present a pull-out resistive configuration within the cancellous bone. The device thereby provides a stable point of attachment for the implant in otherwise-unstable osteoporotic bone.

### BACKGROUND

1. Field of the Invention

The present invention relates to devices for enhancing the effectiveness of Orthopaedic procedures. More particularly, this invention pertains to a device for enhancing the opportunity for successful fixation, and, in turn, outcomes, in procedures that require the attachment of various implants to osteoporotic or cystic bone; these implants may allow fixation of soft tissue (e.g. tendons, ligaments), sutures, or hardware (e.g. metal plates) to osteoporotic bone, as the operative case dictates.

2. Description of the Prior Art

Numerous Orthopaedic procedures require the attachment of tissue, sutures or metal plates to bone. This often involves suturing of connective tissue, such as a tendon or ligament, to a so-called implant that is fixed to the bone. Examples of implants commonly employed for this purpose include suture anchors, screws, plugs and tacks. Also, in fracture care, plates are affixed to bone to promote fracture healing and protect against tensile and shear forces at the fracture site; fully threaded and partially threaded metal screws are often used implants for plate fixation to bone.

The success of the procedure requires that the implant succeed in maintaining secure contact between the attached tissue and bone, or the bone and plate, throughout the healing process. This requires that the implant maintain durable affixation to the bone.

Bone tissue consists of relatively hard outer cortical bone overlying an interior of relatively soft cancellous bone. Fixation of an implant requires that the cortical bone provide a reliable medium of attachment. Many procedures fail or are subject to failure as a result of the poor quality of bone tissue. A common cause of failure, especially prevalent in the elderly, is the presence of soft or osteoporotic bone. A patient with such a condition subjects implant attachment to failure, often leaving cortical defects or holes within the bone as the implant is pulled away.

A known method for securing an implant to otherwise-inadequate bone is the application of cement to augment adhesion between the bone and the implant. Cement is not biodegradable and generates an exothermic heat reaction that can cause necrosis of the surrounding tissue. Further, cement is not easily applied to small holes, especially in arthroscopic procedures where water under pressure with flowing current is employed.

### SUMMARY OF THE INVENTION

The present invention addresses the preceding and other shortcomings of the prior art by providing a bone augmentation device for anchoring an orthopaedic implant to bone tissue. Such device includes a body having proximal and distal sections. The proximal section is of annular shape with the distal section comprising a plurality of arcuate lower segments.

The plurality of arcuate lower segments are aligned about a central axis in a closed configuration. Such central axis is aligned with the axis of symmetry of the annular proximal section.

The preceding and other features of the invention are described in a detailed description that follows. Such description is accompanied by a set of drawing figures. Numerals of the drawing figures, corresponding to those of the written description, point to the features of the invention. Like numerals refer to like features throughout both the written description and the drawings.

### BRIEF DESCRIPTION OF THE DRAWINGS

FIG. 1 is a perspective view of a bone augmentation device in accordance with a first embodiment of the invention;

FIG. 2 is a side sectional view of the device of the invention taken at line 2-2 of FIG. 1;

FIGS. 3A through 3E are side sectional views of the operation of a bone augmentation device in accordance with the first embodiment of the invention, side sectional views illustrating the operation of the invention in accordance with a second embodiment and a perspective view of the device in accordance with a third embodiment of the invention respectively;

FIGS. 4A and 4B are perspective views of a bone augmentation device in accordance with a fourth alternative embodiment of the invention and detail of the hinge structure utilized in such embodiment respectively; and

FIGS. 5A through 5D are cross-sectional views for illustrating the operation of a bone augmentation device in accordance with the fourth and a fifth embodiment of the invention.

### DETAILED DESCRIPTION OF THE PREFERRED EMBODIMENT

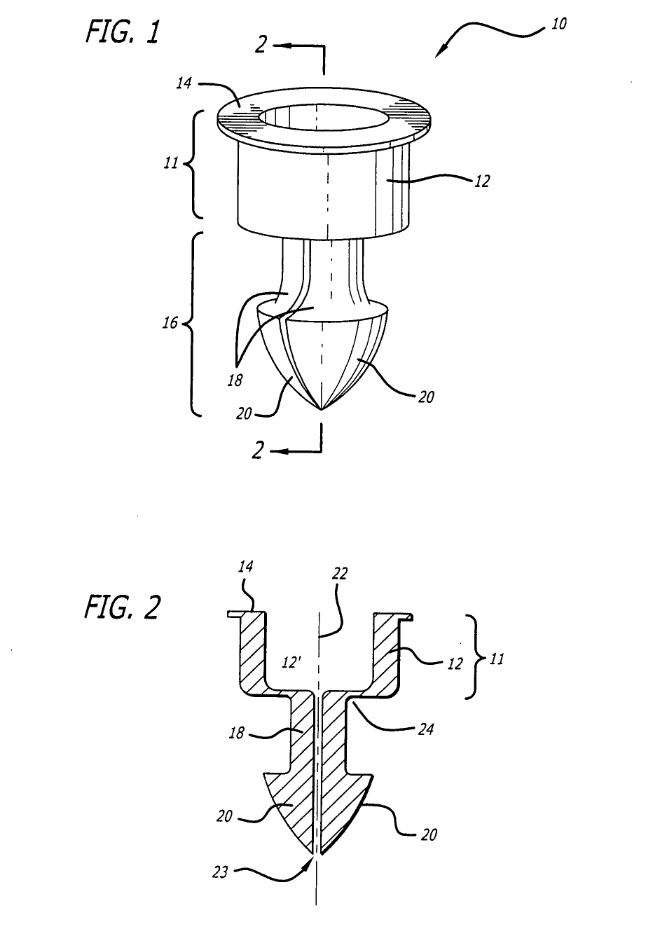

FIG. 1 is a perspective view of a bone augmentation device 10 in accordance with a first embodiment of the invention. The device 10 provides and serves as a means for anchoring an Orthopaedic implant to osteoporotic or other bone that may be inadequate for securely holding the implant for the duration of healing. It may additionally be employed to “save” a procedure that has been compromised by the inability of a patient's cortical bone to hold an implant, often leaving a relatively-large hole in the bone at the point of “attachment”. Thus, the device may salvage the use of the implant, preventing waste.

The device 10 comprises a molded body of relatively non-brittle material, preferably comprising either osteoconductive material, with or without osteoinductive elements adsorbed to, or coating, the surface of the device. Osteoconductive materials provide a biologic scaffold or framework and promote healing by stimulating the formation of bone at the implant site. Osteoinductive chemicals (cytokines) stimulate bone formation by signaling bone-forming cells to generate bone.

Osteoconductive materials include, but are not limited to the following commercially-available compounds: TCP (tricalcium phosphate) and MILAGRO™ beta tricalcium phosphate of Depuy Mitek, Inc. of Raynham, Mass.; TCP/Poly-L-lactic Acid (PLLA) calcium composite of Biocomposites, Ltd. of Staffordshire, England and Wilmington, N.C.; 96 L/4 D Polylactic Acid (PLA) copolymer with beta-MATRY(X)™ of ConMed Linvatec of Largo, Fla.; TCP/PLA BIOCRYL™ of Depuy Mitek, Inc.; CALAXO™ of Smith & Nephew, Inc. of Andover, Mass.; and STERLING® Biologic Matrix of Regeneration Technologies, Inc. of Alachua, Fla.

Suitable osteoinductive materials include, among others, bone morphogenetic proteins (BMPs), platelet derived growth factor (PDGF), fibroblast growth factors (FGFs), parathyroid hormone-related peptide (PTHrp), and transforming growth factor-beta (TGF-B). An example of a commercially available BMP is OP-1® manufactured and marketed by Stryker Biotech of Hopkinton, Mass.

Other suitable materials for forming the device include metal alloys, titanium, cobalt-chrome and steel as well as bioabsorbable materials such as PLA, PLLA and the compound commercially available from Arthrotek, Inc. of Warsaw, Ind. under the trademark LACTOSORB-L15-Copolymer.

The various abovementioned materials offer various advantages that will become further apparent from the discussion that follows. For example, osteoconductive materials utilize biocompatible foreign bodies to form matrices (scaffolds) for accommodating and promoting bone growth while osteogenic materials comprise biologics made of signaling molecules (cytokines) that stimulate bone growth by signaling cells to generate bone tissue. Metal alloys and bioabsorbable materials are also suitable to secure an Orthopaedic implant the requisite time necessary for healing, for example, between soft tissue and bone or between bone and bone (in the case of application of a plate).

Returning to FIG. 1, the device 10 comprises a molded body that includes a proximal section 11 defined by an annular wall 12 having a flange 14 at its upper edge. A distal section 16 of the device 10 comprises a plurality (preferably two or four) of lower segments 18, each of which includes a tapered free end 20. As will be discussed below, the distal section 16 is arranged to be driven from a “closed” configuration (illustrated in FIG. 1) to an “open” configuration by the insertion of a surgical implant. It will be appreciated below that, upon rotation, the tapered free ends 20 of the lower segments 18 act as “barbs” for anchoring the device 10 within bone.

Referring to FIG. 2, a side sectional view of the bone augmentation device 10 taken at line 2-2 of FIG. 1, one can see that the lower segments 18 are aligned symmetrically about a common axis 22 to form the closed configuration of the distal section 16. Such axis coincides with the axis of symmetry of the annular proximal section 11. In the closed configuration, the lower segments 18 may, in the alternative, contact one another or be spaced apart to form an internal vertical channel 23 as shown in FIG. 2. As will be seen below, the lower segments 18 coact with a surgical implant. Accordingly, the size of such features of the device 10 may vary in accordance with the particular type of implant to be utilized by the surgeon.

The views of FIGS. 1 and 2 portray the device 10 with the distal section 16 closed for insertion into a pilot bone hole or defect. The tapered shapes of the free ends 20 of the lower segments 18 facilitate such insertion. As mentioned, once a surgical implant has been inserted into a bone-mounted device 10, the tapered free ends 20 act as barbs that engage the endosteal side of the cortical bone surface.

Radially-directed flexures 24 join the annular wall 12 of the proximal section 11 to the lower segments 18. As mentioned earlier, the device 10 may be molded, for example, of relatively non-brittle material whereby the flexures 24 will not fracture, but rather bend (i.e., rotate radially outwardly), in response to the application of appropriately-directed force. In addition, as shown in FIG. 2, the flexures 24 may be of lesser thickness, and therefore greater flexibility, than are the annular wall 12 of the proximal section 11 and the lower segments 18 of the distal segment 16 of the device 10.

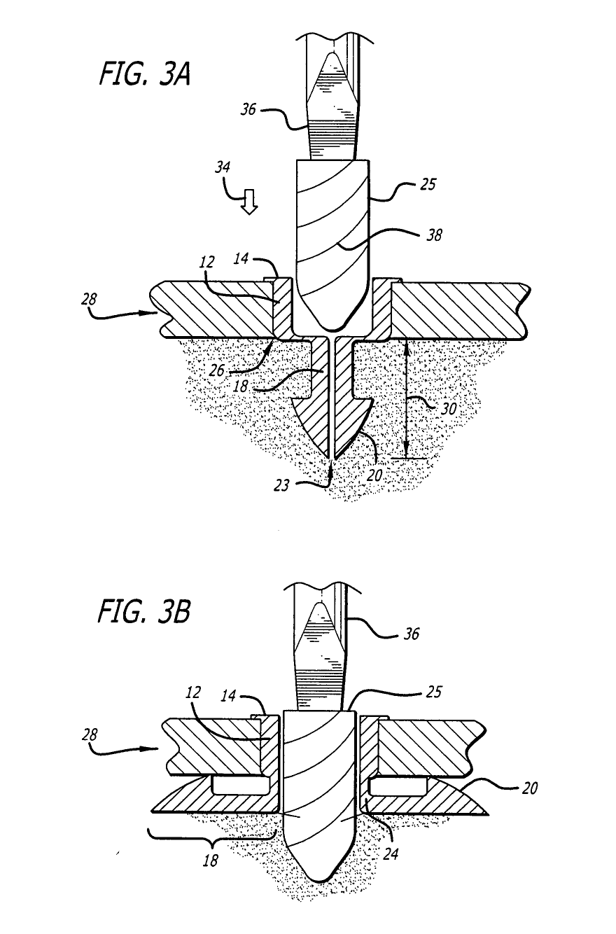

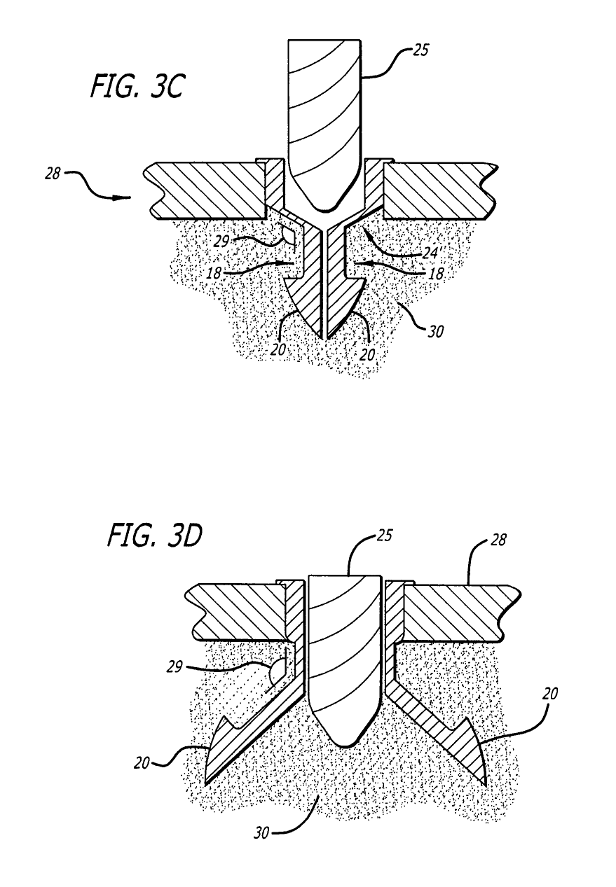

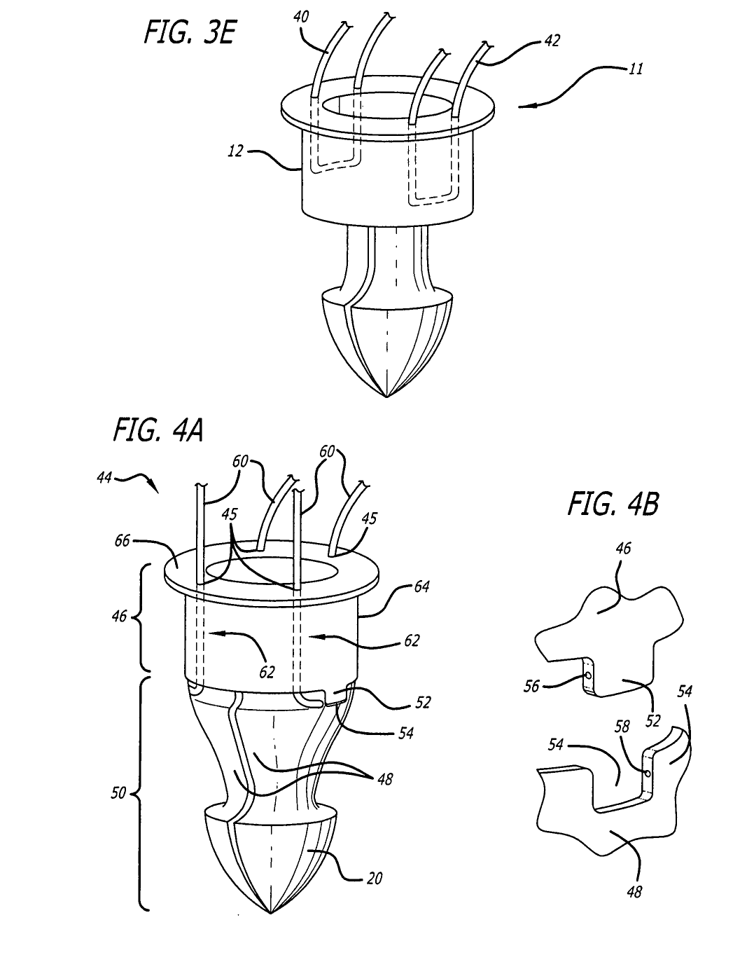

FIGS. 3A and 3B illustrate the operation of the bone augmentation device 10 of FIGS. 1 and 2 for seating a surgical implant (e.g., suture anchor, screw, plug or tack) within bone. Features of the device 10 are indicated with like numerals to those employed in the earlier-described figures. The device 10 is shown in FIG. 3A after insertion into a pilot hole or defect 26 opened within osteoporotic cortical bone 28. The device 10 may be “pre-loaded” or combined with an orthopaedic implant 25 (such as a screw) prior to insertion whereby the implant 25 itself, with an attached driver 36 are combined to introduce it for seating into osteoporotic bone in a single step as shown. When not pre-loaded with the implant as shown, a common tool, such as an orthopaedic clamp, is used to seat the device 10 into the bone pilot hole or defect 26 prior to accepting the implant for bone affixation in a subsequent process step. Seating is facilitated by the tapered shape of the free end 20 of the device 10 in “closed” configuration that leads the device 10 into the pilot hole or defect 26 within the cortical bone 28 to project into the soft cancellous bone 30 thereunder. At the same time, the retainer flange 14 surrounding the proximal section 12 acts as a “stop”, providing feedback to the surgeon with regard to when the device 10 is in place and secure on the surface of the cortical bone 28 and ready to accept the implant for seating into the bone tissue.

The surgical implant (screw) 25 is guided in a downward direction 34 toward and into the hollow interior of the proximal section 12 of the device 10 by the driver 36 that, as mentioned above, is fitted (temporarily) to it. FIG. 3B illustrates the configuration of the device 10 subsequent to application of downward (and rotational) force by the driver 36. (Note: The thread 38 of the screw (employed as the implant 25) is received at the interior of the device 10 rather than by the surrounding osteporotic cortical bone 28.) The downward advance of the implant 25 from the proximal section 11 into the distal section 16 results in the outward rotations of the lower segments 18 about their flexures 24, effectively opening the distal section 16. Such rotations occur in response to the relatively-small internal separation distances (substantially less than the width of the implant 25) between the lower segments 18 when the distal section 16 is closed.

Upon rotation through approximately 90 degrees, the tapered ends 20 of the lower segments 18 now act as “barbs”, engaging the endosteal side of the cortical bone 28. As a result, the device 10 as configured in FIG. 3B securely grips the opposed surfaces of the cortical bone 28 at the lip 14 (exosteal surface) and at the tapered free ends 20 of the lower segments 18 (endosteal surface).

The implant 25 is thus fixed to an augmentation device 10 that, in turn, firmly engages the osteoporotic cortical bone 28. A suture, fabricated with the implant 25, may now be passed through injured tendon or ligament for soft-tissue fixation and apposition to bone. Common implants for fixing wires, sutures, tendons, ligaments and plates to bones can be made of metal or metal alloy, bioabsorbable, osteoconductive or “osteogenic” materials. The device 10 can be of any biocompatible material since, once fixation has been achieved and appropriate healing occurred, the device 10 is rendered obsolete. In the event the bone augmentation device 10 is bio-degradable or made of the same material as the implant 25, the same advantages afforded by the implant for having bio-degradable properties would apply to the implant-with-bone augmentation device combination as there would then exist no difference biologically between the implant and the bone augmentation device. (Note: It is mandatory that the implant and bone augmentation device be of the same metal when a metal implant is employed with a bone augmentation device of metallic composition to prevent the possibility of corrosion-inducing reactions. In contrast, a metal implant may be paired with a device of osteoconductive fabrication without risk of reaction-induced corrosion.)

While the discussion has proceeded to this point with regard to a device 10 in which the lower segments 18 comprising the distal section 16 are hinged to the proximal section 11 so that, upon insertion of an implant 25, the lower segments 18 rotate to a perpendicular orientation with tapered ends 20 engaging osteoporotic cortical bone 28, a device in accordance with the invention may also function successfully without intimate contact between the lower segments 18 and the endosteal side of the cortical bone 28. FIGS. 3C and 3D are side sectional views of a device in accordance with a second embodiment of the invention that parallel FIGS. 3A and 3B. The embodiment of these figures differs from that of FIGS. 3A and 3B insofar as flexures 24′ form an angle 29 with the elongated lower segments 18 of the distal section 16. As a result, upon insertion of an implant 25, the lower segments 18 rotate to final attitudes (illustrated in FIG. 3D) wherein their tapered ends 20 lie within the relatively soft cancellous bone 30 and do not reach or contact the overlying cortical bone 28.

The embodiment illustrated in FIGS. 3C and 3D points to the fact that contact with the cortical bone 28 is not necessary for successful operation of the invention. The device 10 of the invention provides an anchor for a surgical implant, providing substantial resistance from the pulling out of such implant from osteoporotic bone tissue. For this, it is not mandatory that the lower segments of the distal section directly abut the endosteal side of the cortical bone. Rather, the spreading of the lower segments in response to insertion of a surgical implant results in enhancement of pull-out strength. The embodiment of FIGS. 3C and 3D is advantageous in certain circumstances. For example, the angled orientation of the flexure 24′ may be superior in some instances to accept an implant of compatible shape (note: surgical implants are often tapered). Additionally, certain materials may be employed for fabrication of a device that would otherwise be ruled out as overly-brittle to support a ninety-degree opening of the lower segments.

FIG. 3E is a perspective view of a third embodiment of the device of the invention that differs from the preceding embodiments insofar as sutures 40, 42 are molded within the sidewall 12 of the proximal section 11. The presence of such sutures 40, 42 provides the surgeon with a site within the osteoporotic bone for affixing additional tissue during an operation.

FIGS. 4A and 4B are perspective views of a bone augmentation device 44 in accordance with a fourth embodiment of the invention and of the detail of a hinge structure employed therein respectively. Such embodiment functions essentially as the preceding embodiments of integral molded design to anchor a surgical implant within osteoporotic bone. It differs from the integral embodiments in two related aspects. First, unlike the previously-described embodiments, that of FIGS. 4A and 4B consists of three or more separate parts (a proximal section and at least two lower segments of a distal section). Secondly, in the present embodiment the separate parts are hinged together rather than joined by flexures.

Referring to FIG. 4A, the bone augmentation device 44 comprises a proximal section 46 that is joined to lower segments 48 of a distal section 50. As in the prior embodiments, the lower segments 48 of the distal section 50 are caused to rotate outwardly about a hinge mechanism when embedded in bone. Lower segment 48 and distal section 50 are tapered to facilitate placement within a bony defect. Such configuration of lower segments 48 (preferably two or four are employed in both the present and the prior embodiment of the invention) affords the same functional advantages as those of the prior embodiments, namely (1) facilitating entry into the bone pilot hole or defect and (2) acting as a barb to stabilize and fix the device 44 with respect to the cortical bone.

FIG. 4B is a detailed exploded perspective view of a hinge for rotatably joining the proximal section 46 to a lower segment 48 of the distal section 50. Referring to FIGS. 4A and 4B, the hinge comprises two parts. A downwardly-directed tab 52 that is mated with a notch 54 in the upper edge of the free end of a lower segment 48. A channel that continues through the upper edge of the lower segment 48 and the tab 52 at the lower edge of the proximal section 46 is evidenced by the presence of visible openings 56, 58 in the tab 52 and in the upper edge of the lower segment 48 respectively. Such continuous channel is provided for receiving a suture 60 (alternately a dowel, wire or other elongated fastening means) that secures the tab 52 within the notch 54 in rotatable relation.

Referring to FIG. 4A, suture-receiving vertical channels 62 may be formed within the molded annular wall 64 of the proximal section 46 to receive sutures 60 whereby each continuous suture 60 (which continues into the upper portion of a lower segment 48 of the distal section 50, then through the continuous channel, described above, that enables the tab 52 at the lower edge of the proximal section 46 to be rotatably joined to the upper region of the lower segment 48) will offer free ends that exit the flanged upper surface of the annular wall 64. Such location of the free ends of the sutures 60 permit the surgeon to prevent excessive or uncontrolled depth penetration (in a sense, offering a safety net in the event the cortical bone fails to support the flange 66 that is directed outwardly from the upper edge of the annular wall 64 of the proximal section 46) and placement of the device 44 and allow further tissue fixation, if desired.

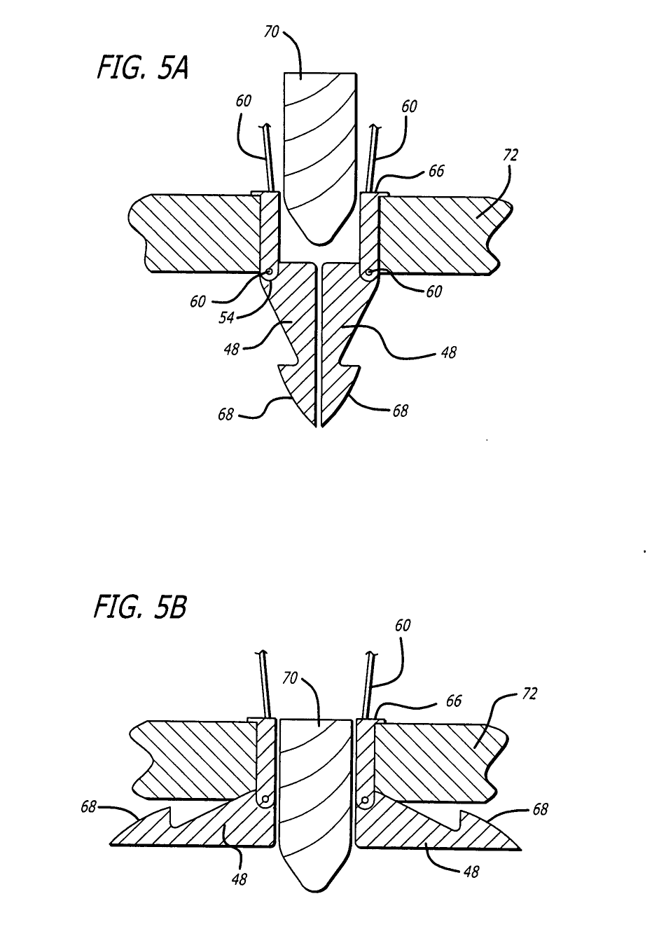

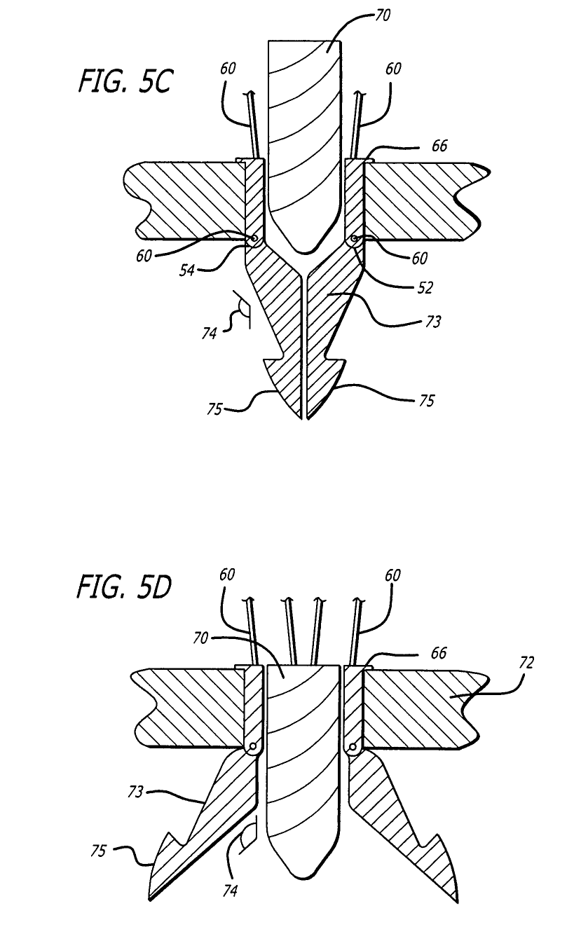

FIGS. 5A and 5B are side sectional views of the device illustrated in the preceding figure in closed and open configurations respectively. It will become clear from the discussion below that the generalized operation of the bone augmentation device 44 closely parallels that of the integral molded devices discussed above. Accordingly, one will appreciate that much of the discussion accompanying FIGS. 3A and 3B applies equally and analogously to that accompanying the views of FIGS. 5A and 5B.

As mentioned earlier, the bone augmentation device 44 functions essentially as that of prior embodiments. In the closed configuration of FIG. 5A, multiple lower segments 48, are aligned as shown. In the closed configuration, the free ends 68 of the multiple lower segments 48 form, in composite, a tapered head for entry into a bone pilot hole or defect. FIG. 5B illustrates the configuration of the bone augmentation device 44 after downward travel of an implant 70, such as a screw (other examples, suture anchor, plug, tack), having a transverse diameter that exceeds the distance, if any, between the opposed interior surfaces of the lower segments 48. As in the case of prior embodiments, the lower segments 48 of the distal section 50 are forced outwardly, pivoting about the hinges that join the lower segments 48 to the proximal section 46. In the open configuration of FIG. 5B, achieved upon the penetration of the orthopaedic implant 70, a vertical channel is now open through the device 44 by the induced separation of the lower segments 48 that accommodates the transverse dimension of the implant as in FIG. 5B. The free ends 68 of the lower segments 48, having been caused to rotate approximately ninety degrees, now act as barbs, making contact with the endosteal side of the cortical bone as shown in FIG. 5B. As in the case of the prior embodiment, the device 44 is thus fixably located within the osteoporotic bone, the barbs at the tapered free ends 68 and the flange 66 about the top of the proximal section 46 combining to secure the opposed surfaces of the cortical bone 72.

FIGS. 5C and 5D illustrate a fifth embodiment of the invention in cross-section. This embodiment incorporates the hinge of the prior embodiment in which a suture 60 is passed through channels formed within a proximal section 46 and within a tab 52-and-notch 54 arrangement to rotatably secure lower segments 73 of a distal section to the proximal section 46. In contrast to the prior embodiment, the fifth embodiment utilizes lower segments 73 that include interior surfaces which define an interior angle 74. The presence of such interior angle 74 causes the fifth embodiment of the device to function in a similar manner to the operation of the third embodiment illustrated and described with reference to FIGS. 3C and 3D above. That is, the angled interior surfaces 74 of the lower segments 73 of the device of the fifth embodiment cause such device to open upon insertion of an implant 70 in approximately the same way that the orientation of the flexure 24′ of the device of the third embodiment at an angle 29 causes that device to open. As can be seen in FIG. 5D, the presence of the angled interior surfaces of the lower segments 73 results in the tapered ends 75 of the segments 73 lying within cancellous bone that lies beneath or within the cortical bone 72. As mentioned earlier, such a configuration is useful, and, in some cases, preferable for preventing undesired implant pull-out.

The bone augmentation device may be fixed to bone prior to insertion of an implant or, in the alternative, it may be paired as a unit with the implant as illustrated.

By utilizing a bone augmentation device in accordance with the invention, an Orthopaedic surgeon is enabled to perform procedures that would otherwise fail or be subject to failure during the course of healing secondary to osteoporotic, soft or cystic bone. The device further enables the redoing of failed procedures that would otherwise be subject to abandonment by salvaging the utility of a failed implant, thus avoiding waste of implant resources. As a result, patients, especially the elderly who are more commonly subject to osteoporosis, can obtain the benefits of otherwise-unavailable remedial medical procedures. The resultant increases in limb motion offers the possibility of dramatic lifestyle enhancement.

While this invention has been described with reference to its presently preferred embodiments, it is not limited thereto. Rather, the invention is limited only insofar as it is defined by the following set of patent claims and includes within its scope all equivalents thereof.

What is claimed is:

1. A bone augmentation device for anchoring an orthopaedic implant to bone tissue comprising, in combination:
a) a body having proximal and distal sections;
b) said proximal section being of annular shape;
c) said distal section comprising a plurality of arcuate lower segments;
d) said plurality of arcuate lower segments being aligned along a central axis in a closed configuration; and
e) said central axis of said distal section being coaxial with the axis of symmetry of said annular proximal section.

2. A bone augmentation device as defined in claim 1 further including a tapered barb adjacent a free end of each of said lower segments.

3. A bone augmentation device as defined in claim 2 wherein said annular proximal section includes an outwardly-directed flange.

4. A bone augmentation device as defined in claim 3 wherein said outwardly-directed flange projects from an upper edge of said annular proximal section.

5. A bone augmentation device as defined in claim 1 wherein said proximal section is integrally molded with said distal section.

6. A bone augmentation device as defined in claim 5 further including a flexure joining said proximal section to each of said lower segments of said distal section.

7. A bone augmentation device as defined in claim 5 wherein each of said flexures is substantially orthogonal to an annular wall of said proximal section and to the length of a lower segment of said distal section.

8. A bone augmentation device as defined in claim 5 wherein each of said flexures is angularly-inclined with respect to an annular wall of said proximal section and to the length of a lower segment of said distal section.

9. A bone augmentation device as defined in claim 1 further including at least one suture molded within said proximal section with free ends projecting from an upper edge of said proximal section.

10. A bone augmentation device as defined in claim 4 wherein each of said lower segments is joined to said proximal section by a hinge.

11. A bone augmentation device as defined in claim 10 wherein each of said hinges comprises:
a) a tab that projects downwardly from said proximal section; and
b) a notch for receiving said tab formed within a mating lower segment.

12. A bone augmentation device as defined in claim 11 wherein said hinge further includes:
a) a channel through said tab;
b) channels in said lower segment at opposed edges of said notch, each of said channels being aligned with said channel through said tab; and
c) an elongated fastener being received within said channels.

13. A bone augmentation device as defined in claim 12 wherein said elongated fastener comprises a suture.

14. A bone augmentation device as defined in claim 12 wherein said elongated fastener comprises wire.

15. A bone augmentation device as defined in claim 12 wherein said elongated fastener comprises a dowel.

16. A bone augmentation device as defined in claim 10 wherein inner edges of said lower segments are aligned with said central axis along their entire lengths.

17. A bone augmentation device as defined in claim 10 wherein the inner edges of said lower segments include angularly-inclined portions.

18. A bone augmentation device as defined in claim 17 wherein said lower segments are symmetrically aligned so that said angularly inclined portions of said inner edges of said lower segments are adjacent said hinges joining said lower segments to said proximal section.

19. A bone augmentation device as defined in claim 1 comprising biocompatible material.

20. A bone augmentation device as defined in claim 1 comprising metallic material.
